(19) United States
(12) Patent Application Publication
(10) Pub. No.: US 2012/0289780 A1
(43) Pub. Date: Nov. 15, 2012

(54) INTRALUMEN MEDICAL DELIVERY VESSEL PROPELLED BY SUPERCONDUCTIVE REPULSION-LEVITATION MAGNETIC FIELDS

(76) Inventors: Laszlo Farkas, Ojai, CA (US); Leslie Farkas, Ojai, CA (US)

(21) Appl. No.: 13/505,724

(22) PCT Filed: Nov. 17, 2010
(86) PCT No.: PCT/US2010/002996

§ 371 Date: Jun. 8, 2012

Related U.S. Application Data
(60) Provisional application No. 61/262,100, filed on Nov. 17, 2009.

(51) Int. Cl. A61M 5/00; A61B 1/04

(52) U.S. Cl. 600/109; 604/93.01; 604/131; 604/285

(57) ABSTRACT

A magnetically guided device driven by the repulsive forces generated by superconductive materials, housed in a thermally insulated vessel, due to phenomenon known as the Meissner-effect in response to the externally generated magnetic fields. The vessels will be installed in or on medical diagnostic, delivery or other procedural devices or capsules, and will enable wireless maneuvering and navigation of the host device through the lumens and cavities of the human body without any physical contact. Medical application fields include, but are not limited to, visual mapping, diagnostics, biopsy and other therapeutic and drug delivery procedures in the human body. The vessel is equipped with superconductive material, such as superconductive rings and/or disks, possessing supermagnetic properties. Shaped externally generated magnetic fields exert sufficient magnetic forces and rotational torques on the superconductive material causing the host device to move, tilt and rotate in the body lumens and cavities following the operator's closed-loop regulated directional and orientation commands.

### RELATED APPLICATION DATA

Applicant claims the benefit of and priority to U.S. Provisional Patent Application No. 61/262,100, filed Nov. 17, 2009.

### BACKGROUND

1. Field of the Invention

This invention relates to medical devices, and more particularly to magnetically guided freely moving medical devices deployed to move within the lumens, cavities and chambers of the human body.

2. Description of the Related Art

Ingestible diagnostic, delivery and therapeutic devices, such as ‘GI capsules’, traveling through the cavities and ducts of the gastrointestinal tract, have been in use since year 2001. When the patient swallows such pill, the natural muscular (peristaltic) movement of the digestive tract propels it through the intestine lumen. While the capsule is moving through the intestine lumen, a small camera enables the physician to inspect the walls of the intestinal ducts for possible detection of tumors, ulcers or bleeding. However, the speed, position and the direction of the capsule and the small camera within the capsule are uncontrolled. Obtaining and maintaining a desired observational point or viewing direction are impractical, and most of the intestinal walls remain uninspected during a single passage. Returning and delivering drugs to a specific locale is imprecise and mostly unattainable.

Manually operated devices of endoscopy and colonoscopy have limited success to reach clinically important anatomic sites, and generally do not enjoy patients' acceptance. With the rapid increase of cases of stomach ulcers and colon cancers, effective and painless methods of regular preventive and investigative examinations are needed. The supermagnetic propulsion vessel of this disclosure offers a non-contacting, controlled procedure eliminating the control instability issues associated with magnetically operated un-tethered device navigation, and enables rapid anatomic site acquisitions for location-specific diagnoses and treatments.

Therapeutic drug delivery to organs, such as the brain, the heart, the kidneys and other critical organs have similar difficulties in reaching the sites of diagnosis and therapeutic interest. Freely moving delivery capsules for deployment through the urinary ducts or the cardiovascular lumens become possible by using the supermagnetic propulsion vessel which can be levitated-suspended, moved or held in place by non-contacting external magnetic fields.

### SUMMARY

In one embodiment, as embodied and broadly described herein, a device is disclosed that is adapted to be magnetically guided due to superconductive material exhibiting supermagnetic properties. The superconductive material is contained within a thermally insulated vessel and the device can be .maneuvered using supermagnetic propulsion in response to externally generated magnetic fields. The superconductive material is advantageously positioned within the thermally insulated vessel and can be in the form of a ring, disk, plate, or other shape. Moving and directing the device is accomplished by utilizing the superconductive Meissner-effect which repels these elements in response to externally generated magnetic fields. Generating the external fields with the proper direction and magnitude relative to these supermagnetic elements, will levitate, suspend, move and orient the vessel in a stable and controllable manner. Holding the vessel in place is achieved by controlling the external magnetic fields such that the repulsive Meissner diamagnetic forces balance on the superconductive material against the weight of the device and against the various forces holding or affecting the device within the patient. Moving and directing the capsule is accomplished by electronically shaping and moving the externally generated magnetic field in relation to the capsule utilizing a variety of core-coil electromagnets suitable to produce such variable fields.

In another embodiment of the invention, the device comprises electronic equipment, such as but not limited to, a camera to take pictures and/or record video of its surroundings as well as a wireless transmitter to transmit the captured pictures and/or recorded video to an external receiver. The device further comprises a light source to illuminate the environment.

In yet another embodiment of the invention, the device comprises medical equipment, such as but not limited to, an injection or spray mechanism to administer a drug or reactive agent, or diagnostic equipment adapted to collect a sample of its environment.

These and other aspects and advantages of the invention will become apparent from the following detailed description and the accompanying drawings which illustrate by way of example the features of the invention.

### BRIEF DESCRIPTION OF THE DRAWINGS

FIG. 1 is a perspective view of an embodiment of the capsule according to the invention.

FIG. 2 is a perspective view of the capsule of FIG. 1 showing portions which are internal to the capsule.

FIG. 3. is a cross-sectional view of an embodiment of the capsule according to the invention.

FIG. 4 is an exploded view of an embodiment of the capsule according to the invention.

FIG. 5. is a perspective view of an embodiment of the capsule according to the invention

FIG. 6 is a cross-sectional view of an embodiment of the capsule according to the invention.

FIG. 7 is a cross-sectional view of an embodiment of the capsule according to the invention.

FIG. 8 is a cross-sectional view of an embodiment of the capsule according to the invention.

FIG. 9 is a cross-sectional view of an embodiment of the capsule according to the invention.

FIG. 10 block diagram of the system used to magnetically guide the capsule according to the invention.

FIG. 11 is a perspective view of the system used to magnetically guide the capsule according to the invention.

FIG. 12 is the flux diversion due to diamagnetic properties of a material M.

FIG. 13A and 13B show the magnetic fields for movement in the ‘Z’ direction.

FIG. 13C is a graph of the B field strength of FIGS. 13A and 13B.

FIG. 14A shows the magnetic fields for movement in the direction.

FIG. 14B is a graph of the B field strength of FIG. 14A.

FIG. 15A shows the magnetic field for movement in the ‘X’ and ‘Z’ direction.

FIG. 15B is a graph of the B field strength of FIG. 15A.

### DETAILED DESCRIPTION

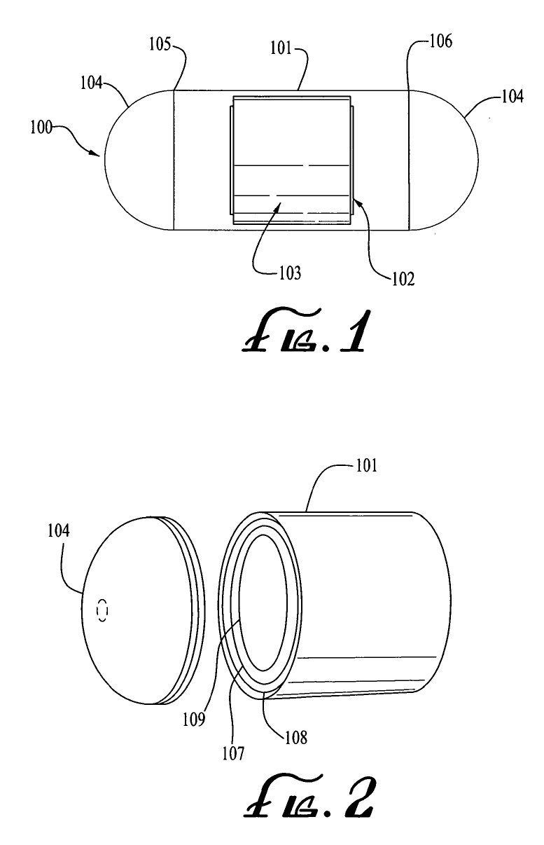

Embodiments of the invention provide an improved medical device adapted to be magnetically guided within the lumens, cavities and chambers of the human body. Referring to FIG. 1 a capsule 100 incorporating features of the invention includes a housing 101, a vessel 102, an insulation material 103 and enclosures 104. The housing 101 comprises a first end 105 and a second end 106, wherein the first end 105 and second end 106 each are adapted to receive an enclosure 104. In a preferred embodiment, the housing 101 is cylindrically shaped and is adapted to receive vessel 102, such that vessel 102 is within the housing 101. However, the shape of housing 101 is not limited to a cylinder; housing 101 can also be shaped in the form of an ellipse, sphere, or any other shape. Additionally, the enclosures 104 of FIG. 1 are shown as being dome-shaped, but can also be formed of many different shapes.

In an embodiment incorporating features of the invention, the vessel 102 comprises superconductive material 107, wherein the superconductive material 107 comprises at least one of superconductive rings, disks, plates, domes or a combination thereof, such that the superconductive materials 107 have supermagnetic properties. The shape of the superconductive materials 107 is not limited to the shapes listed, but can be any shape. In superconducting materials, the characteristics of superconductivity appear when the temperature of the material is lowered below the critical temperature. In an embodiment of the invention, the superconductive materials 107 are cryogenically cooled in order to attain superconductivity and to freeze the trapped magnetic fields into the superconductor. However, other cooling methods known in the art may be used to lower the temperature of the superconductive materials 107 below the critical temperature. The superconductive materials 107 can be made of anisotropic High Temperature Superconductor (HTS) materials, such as yttrium barium copper oxide (YBCO) or other superconductor materials known in the art.

The insulation material 103 provides heat transfer insulation to the capsule 100 such that the temperature increase of the superconductive materials 107 from a pre-cooled temperature to the critical temperature takes a few hours. During this time, the capsule 100 will exhibit the supermagnetic effects and will continue to be magnetically guided. In one embodiment of the invention, the insulation material 103 provides sufficient heat transfer insulation such that the capsule 100 retains superconducting characteristics for at least fifteen (15 ) minutes. In yet another embodiment, the insulation material 103 provides sufficient heat transfer insulation such that the capsule 100 retains superconducting characteristics for at least thirty (30 ) minutes. When the capsule 100 no longer exhibits superconducting characteristics, the natural peristaltic movements will excrete the capsule 100 in due time.

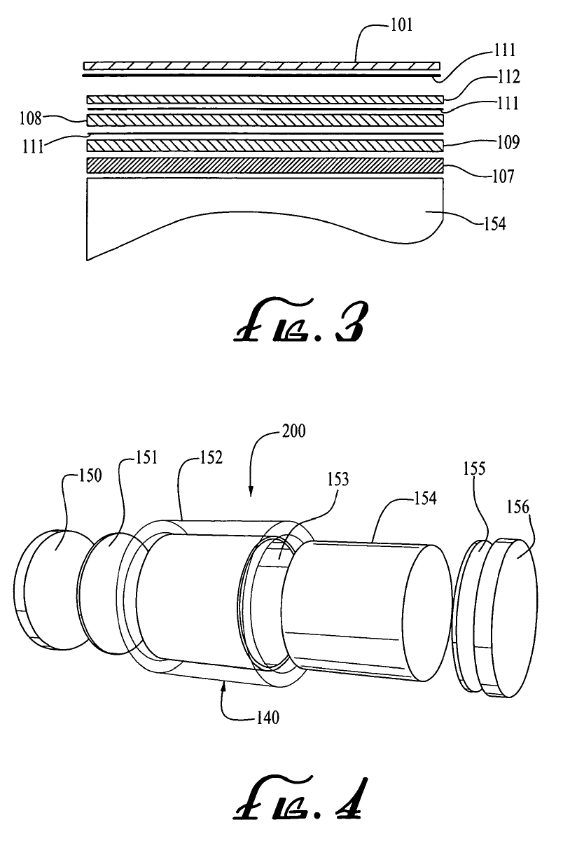

In embodiments of the invention, the insulation material 103 is configured to comprise a plurality of insulation layers, such as but not limited to, a plurality of Mylar® layers covered by aluminum mirror layers. However, other very low thermal-conductivity insulation layers known in the art can be used instead of Mylar® for the insulation material 103. Referring to FIG. 2, the insulation material 103 comprises an outer insulation jacket 108 around the outer surface of superconductive material 107 and an inner insulation jacket 109 around the inner surface of the superconductive material 107. In the embodiment of the vessel 102 of FIG. 2, the superconductive material 107 is covered by the insulation jackets 108, 109 such that the superconductive material 107 is interposed between the inner and outer insulation jackets 108, 109. In another embodiment, such as FIG. 3, a plurality of insulation jackets 108, 109 can be on the outer surface of the superconductive material 107, wherein a thermal plug 154 is disposed within the inner surface of the superconductive material 107 to further provide heat transfer insulation. In embodiments of the invention, the insulation jackets 108, 109 are formed of a plurality of Mylar® layers comprising an aluminum mirror layer coating on the outer surfaces of the plurality of Mylar® layers, such that the plurality of Mylar® layers are interposed between the aluminum mirror layers. The aluminum mirror layers coating the plurality of Mylar® layers reflect heat in order to minimize heat transfer. This insulation technique is effective in minimizing heat transfer for conductive, radiated and convectional heat penetration. Other insulation techniques may be used depending on the required procedure time and the critical temperature of the superconductive material. In some embodiments, the vessel 101 is impregnated into a single vacuum insulated unit, such as but not limited to a Dewar.

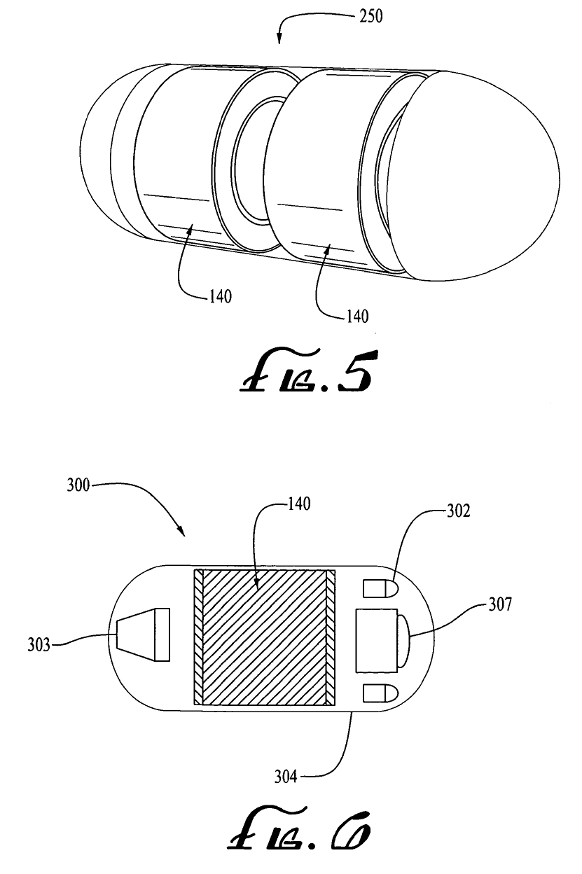

FIG. 4 discloses another embodiment of a capsule 200. In this embodiment the capsule 200 (not shown) comprises a vessel 140 comprising a superconductive cylinder 153 and a plurality of superconductive disks 151, 155. In some embodiments of the invention, the assembled vessel 140 can be approximately 11 mm in diameter and 10 mm in length. However, the vessel 140 and capsule 200 can have different dimensions depending on the procedure to be conducted and/or where the capsule is to be deployed. The superconductive cylinder 153 and disks 151, 155 have a thickness of about 0.25 mm to 0.50 mm, limited only by physical strength for manufacturing and handling. The vessel 140 further comprises an outer insulation jacket 152 that covers the outer surface of superconductive cylinder 153 and a thermal plug 154 which is disposed within the superconductive cylinder 153. The thermal plug 154 is made of a light-weight and high thermal capacity material, such as but not limited to aluminum. When pre-cooled as part of the vessel 140 assembly, the thermal plug 154 assists in keeping the temperature of the superconductive cylinder 153 and disks 151, 155 below the critical temperature for an extended period of time. Vessel 140 further comprises insulation disks 150, 156 to thermally insulate the superconductive disks 151, 155. In embodiments of the vessel 140, the insulation is sufficient to provide heat transfer insulation such that the capsule 200 retains superconducting characteristics for at least fifteen (15) minutes; whereas in other embodiments the capsule 200 retains superconducting characteristics for at least thirty (30) minutes. The insulation jacket 152 and the insulation disks 150, 156 in FIG. 4 comprise a plurality of insulating layers coated with mirror layers on the surfaces, such that the plurality of insulating layers are interposed between the mirror layers. The vessel 140 is impregnated into a single vacuum insulated unit. However, other manufacturing and assembly techniques may be used as technology progresses with different kinds of insulation materials. FIG. 5 discloses another embodiment of a capsule 250, which is similarly configured to capsule 200, but instead uses a plurality of vessels 140.

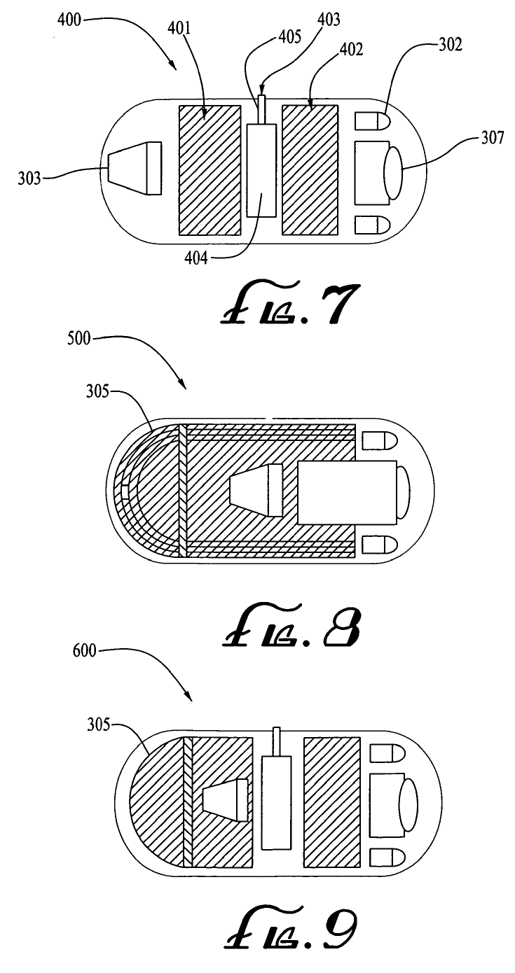

FIG. 6 discloses another embodiment of a capsule 300. In this embodiment, the capsule 300 comprises the vessel 140 disclosed above and in FIG. 4, but further comprises a camera 307, at least one LED light 302 and a video broadcast unit 303. The camera 307, at least one LED light 302 and the video broadcast unit 303 are housing within housing 304 of capsule 300. The vessel 140 is pre-cooled below the critical temperature separately from the housing 304 of capsule 300 and is not inserted into housing 304 until capsule 300 is to be used. The housing 304 is stored at room temperature and the insulation of vessel 140 allows the capsule 300 to exhibit superconducting characteristics as described herein. Furthermore, the insulation of vessel 140 minimizes heat transfer such that the temperature of the vessel 140 does not negatively impact the performance of the camera 307, at least one LED light 302, video broadcast unit 303, or any other pieces of equipment disposed within capsule 300.

FIG. 7 discloses another embodiment of a capsule 400. In this embodiment, the capsule 400 comprises a plurality of vessels 401, 402 which are similar to vessel 140 described above. Capsule 400, similar to capsule 300, comprises a camera 307, at least one LED light 302 and a video broadcast unit 303, but further comprises a therapeutic device 403 which comprises a container 404 to house a drug or reactive agent, and an injection or spray mechanism 405 to administer the drug or reactive agent stored within container 404. In other embodiments, the therapeutic device 403 comprises a retrieval device 405 to collect a tissue sample to be stored within container 404. In yet other embodiments, the capsule 400 can comprise both the injection or spray mechanism and the retrieval device.

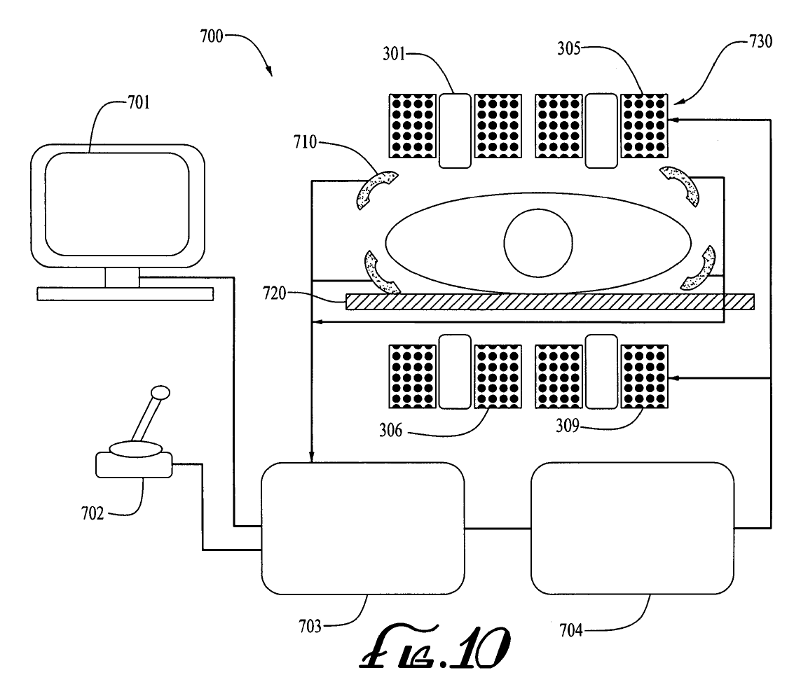

FIGS. 8 and 9 disclose additional embodiments of the invention. The capsule 500 disclosed in FIG. 8 is similar to the capsule 300 of FIG. 6, but further comprises a superconductive dome 305 which is insulated in a similar manner as vessel 140. The capsule 600 disclosed in FIG. 9 is similar to the capsule 400 of FIG. 7, but further comprises the superconductive dome 305.

The capsules described herein are adapted to be magnetically guided due to superconductive materials exhibiting supermagnetic properties. The superconductive materials are contained within the thermally insulated vessel and the capsule can be maneuvered using supermagnetic propulsion in response to externally generated magnetic fields. The superconductive materials respond to externally generated magnetic fields by repelling from the externally generated magnetic fields due to the phenomenon called Meissner-effect. Generating the external magnetic fields with the proper direction and magnitude relative to these superconductive materials, will levitate, suspend, move and orient the capsule in a stable and controllable manner. Holding the capsule in place is achieved by controlling the external magnetic fields such that the repulsive Meissner diamagnetic forces balance on the superconductive material against the weight of the capsule and against the various forces holding or affecting the capsule within the body. Moving and directing the capsule is accomplished by electronically shaping and moving the magnetic loci of the externally generated magnetic fields in relation to the capsule's superconductive characteristics utilizing a variety of core-coil electromagnets suitable to produce such variable magnetic fields. In some embodiments, moving and directing the capsule is accomplished by utilizing the permanent magnet effect of trapped magnetic fields frozen into the superconductive disks, while the superconductive plates allow for axial rotation of the capsule.

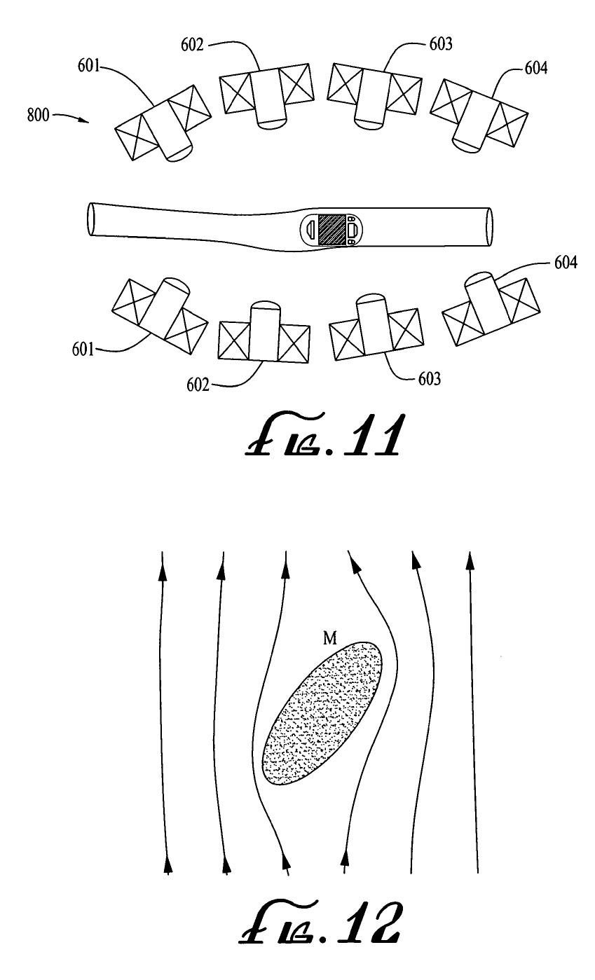

FIG. 10 discloses an embodiment of a system 700 for magnetically guiding the capsule described herein. The system of FIG. 10 comprises a display 701, an input device 702, regulator 703, amplifiers 704, sensor 710, a table 720, and an external magnetic field generator 730. The external magnetic field generator 730 comprises a plurality of electromagnetic coils 301, 305, 306, 309 and is adapted to form and shape a 3D magnetic field around the capsule to form a magnetic gradient valley which holds the capsule by the repulsive Meissner effects. The external magnetic field generator 730 also provides the necessary field strength and gradient to attract or repel the capsule's trapped magnetic field elements. These gradient forces move and orient the capsule. To obtain the desired location and orientation of the capsule, this complex dynamic magnetic field is regulated by a computerized closed loop system comprising the input device 702 for operator input, magnetic and visual feedback from the capsule. In one embodiment, sensors 710 are magnetic feedback sensors which receive polarized high frequency transmissions sequentially transmitted for each of the three capsule axes from the capsule to the sensors 710. Triangulation methods and algorithms are used to compute coordinates in reference to the external electromagnetic structure where the sensors 710 are located. In another embodiment, the capsule has receivers and the sensors 710 become a sequential high frequency broadcast network. The capsule's three-axis sensor signals are then transmitted to an external receiver for decoding and computing the location of the capsule, again in reference to the external magnetic assembly.

In an embodiment of the capsule which has a video camera, the video signal is displayed on display 701 for the operator for man-in-the-loop navigation. The content of the video can be deciphered by image processing and the information used for navigation.

Using the capsule in any of the listed medical procedures requires the patient lying on the table 720 which is surrounded by the external magnetic field generator 730. The pre-cooled insulated vessel will be inserted, by an appropriately automated device, into a room temperature capsule, which in turn will be sealed by the same automated device. The capsule will be swallowed or inserted into the patient. The external magnetic field generator 730, regulator 703 and amplifiers 704 will be activated and the capsule navigation can begin. Sensors 710 indicate the location of the capsule and the externally generated magnetic field and field gradients begin to hold and control the capsule. An operator using input device 702, such as but not limited to a joystick, can direct the capsule as directed by the input device 702.

In one procedure of intestinal investigation each patient has on the average two hours to be examined. This means that in a regular 8 hour work day, 4 procedures can be performed using 4 capsules per day. In one embodiment, using HTS material with critical temperatures at liquid Nitrogen (77K), each table 720 comprises a cryogenic-cooler (not shown) adapted to house a plurality of vessel assemblies. In some embodiments, the cryogenic-cooler needs to keep 4 vessel assemblies at the pre-cooled temperature of approximately 55K for daily use. If the warming up temperature gradient from 55K to 77K is approximately 5K/hour, and the cool-down roughly is also 5K/hour from room temp to the pre-cooled temperature level, it will take approximately 60 hours to get a fully insulated vessel assembly ready to be deployed starting the procedure at 55K. This is approximately 2.5 days for each vessel. Thus, the cryogenic-cooler has to store a minimum of four rows of 4 vessel assemblies, the first row of 4 is ready in the morning of the first day, each vessel sitting at 55K. The fourth row of 4 is loaded in at room temperature and starts to cool down. The third and second rows of 4 and 4 are cooling down with temperatures between room and the critical temperature. Once the first row is empty, the cryogenic-cooler is adapted to rotate the rows. Thus, the minimum number of vessels in the cryogenic-cooler is 16. However, in other embodiments the cryogenic-cooler can be configured to house 5 or more vessels per row depending on factors, such as the patient throughput and/or the length of the workday. This method of revolving vessel-columns cooling down in sequence supplies continuous vessel flow available for every day. There are no electronics or any other power dissipation in the vessels during cool-down.

A number of electromagnetic coil-core configurations are suitable to generate magnetic fields with the necessary field strength and gradient. Electromagnetic coils 301, 305, 306 and 309 are configured around the patient's body having an operating or control region within the human body. To obtain the desired location and orientation of the vessel with 6 degrees of freedom, the fields are generated by independently controlling the coil current magnitudes and polarity from the amplifiers 704.

An important feature of the system of FIG. 10 is that even without the visual feedback, the vessel is controllable in all modes of magnetic influence; the repelling nature of the superconductive Meissner effect is inherently stable. Adding an optional electronic feedback device, such as sensors 710, to the system enables automated guidance control, mapping and the ability to return the capsule to the same site automatically

FIG. 11 discloses another embodiment of a system 800 used to magnetically guide the capsule. In the system 800, two sets of four coil-core electromagnets 601, 602, 603 and 604 surround the table 720 and the patient. The coil-core electromagnets surrounding the patient's body builds a gradient valley sloping toward the capsule location from all directions.

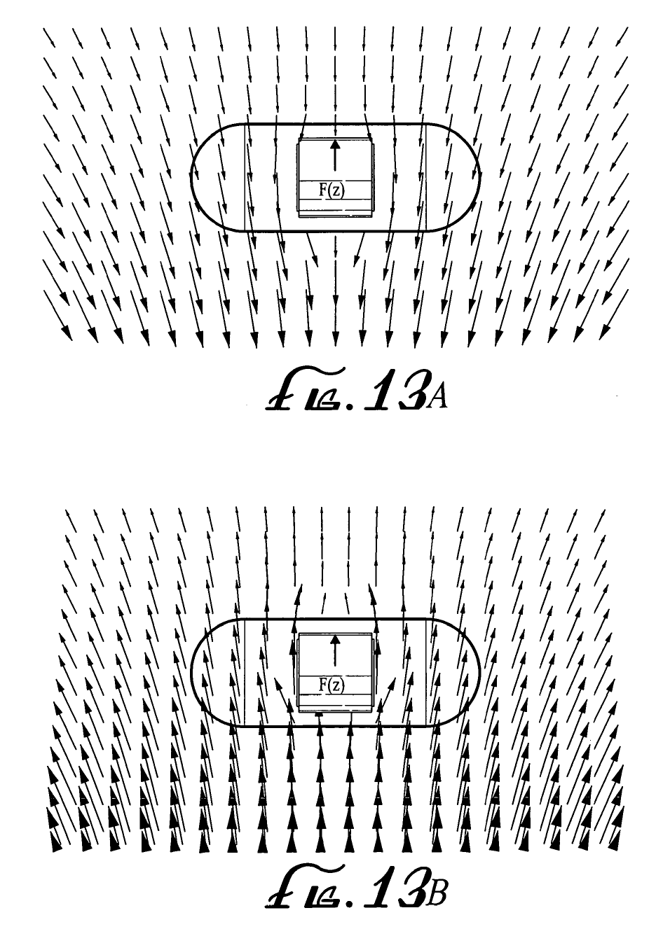

The physics principle underlying the magnetic guidance of the medical device is a unique form of diamagnetism observed in High Temperature Superconductors (HTS) under moderate magnetic field conditions. FIG. 12 shows flux diversion due to diamagnetic properties of material M, which has permeability 0<<μₘ<<1. If M is a superconductor, the superconductor finds an equilibrium state where the sum of electron kinetic and the interior magnetic energies is minimum, which state for the macroscopic supermagnetic body corresponds to the expulsion of magnetic flux. Indeed, as found by F. & H. London in the early 1930s, the flux penetrates into the superconductive material about 500 Å to 2000 Å, a very small thickness indeed. In terms of E energy:

$$E = {E}_{0} + \frac{1}{8 π} \int [ {H}^{2} + {ε}_{L}^{2} {\vert ∇ \times H \vert}^{2} ] d r$$

where H is the magnetic field, ε is the penetration depth and r is the location in a coordinate system in which E₀ is the sum of the electrons energy in condensed state and the kinetic energy of the permanent super-currents. The penetration depth, named after F. & H. London, is:

$${ε}_{L} = \sqrt{[ \frac{m {c}^{2}}{4 π {n}_{s} {e}^{2}} ]}$$

where nₛ=n, the total number of conduction electrons in cubic centimeter. The field configuration in the interior of the HTS, which minimizes the free energy will satisfy the conditions of:

H+[ε_{L}²∇×Δ×H]=0

When combined with the Maxwell equations:

$$∇ \times H = \frac{4 π {j}_{s}}{c} and ∇ \cdot H = 0 ,$$

the field distribution and the currents can be calculated. Here jₛ is the current density in the HTS. The finite solution leads to conclude that the fields will run parallel with the HTS surface and the exerted force will be determined by the field gradient across the external surface of the HTS penetrating into the material with the London depth of:

$${ε}_{L} = \sqrt{[ \frac{m {c}^{2}}{4 π {n}_{s} {e}^{2}} ]}$$

integrated over the entire surface of the HTS. The repelling force exerted on the total HTS surface is:

$${F}_{S} = k \cdot {I}_{coil}^{2} \cdot \int \frac{d B}{d ε} d s \qquad [ 1 ]$$

I is the coil current and B is the field strength generated by the external magnetic field generators at the capsule location, and the integration is over the s surface of the HTS in the capsule.

Practical computations in FEA magnetic simulations for any shapes of HTS surface can proceed based on estimating permeability less than 1.00 for accounting for the HTS Diamagnetic nature. Values of μ≦ 1/1000 produce force calculation errors less than 1%.

Force magnitude of maximum 1 Newton is obtained for a vessel volume of 4.5 mm diameter and 8 mm length. This magnitude is sufficient for controlling capsules, and micro-devices within the human body. However, the vessel walls can be very thin due to the small London penetration depth. Thus, the capsule can be very light. This is an important feature when the medical device needs to carry the useful load of diagnostic and therapeutic equipments. The limitation for larger forces is the maximum H_{c} critical field strength around the HTS, above which the material may be come normal even below T_{c} critical superconductive temperature. All calculations and simulations keep the maximum field strength below 2.5-3.0 kGauss.

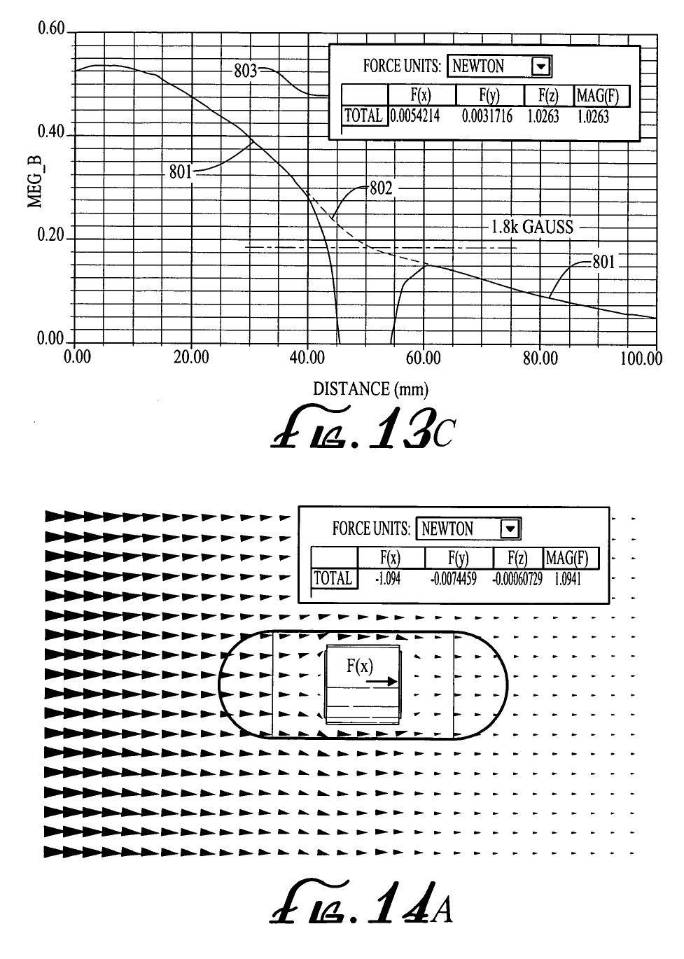

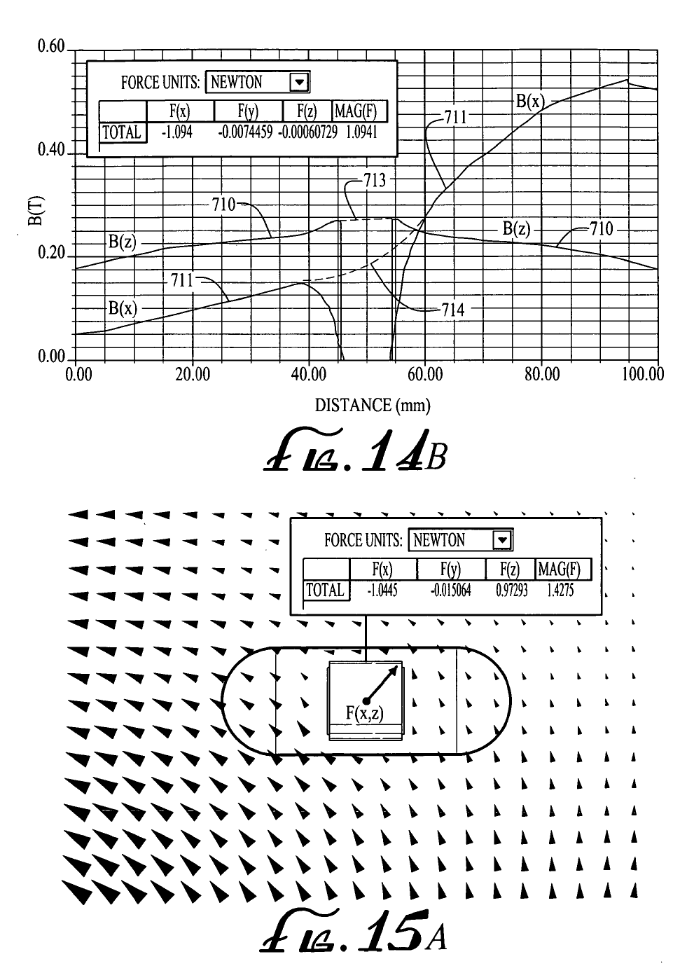

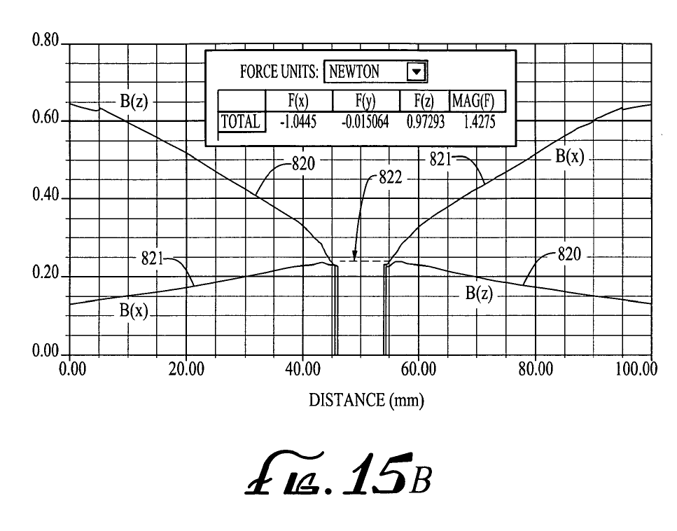

The diamagnetic nature of the HTS materials used in this invention assures that the capsule movement is always along the decaying slope of the field gradient independent of the field vector polarity. The capsule can be levitated in ‘+Z’ direction and moved in the ‘X’ and ‘Y’ directions in a stable manner away from higher absolute value magnetic fields toward the lower absolute values as shown in FIG. 13A, 13B and 13C for levitation, FIG. 14A and FIG. 14B for movement in ‘−X’ direction, and FIG. 15A and FIG. 15B for combined ‘−X’ and ‘+Z’ movements. FIG. 13C is the graph of the B field strength along the Z axis. Between 46 mm and 54 mm the presence of the capsule is evident due to the full expulsion of the flux (B≅0 Gauss). The average Field strength 802 at the location of the capsule would be about 1.8 kGauss, which level is below the H_{c} of 2.5 kGauss. The slope of the fields 801 providing the field gradient in the general force equation [1] is:

$$\frac{d B}{d z} = 4.5 Tesla \text{/} m .$$

Using Ansoft magnetic simulation with μ=0.001, the resulting F(z) force levitating and moving the capsule upward is 1.0263 Newton (803). It is evident from FIG. 13A and 13B that the direction (polarity) of the Z field is of no consequence, the repelling force points upward.

FIG. 14A shows the B field exerting 1.094N force on the vessel moving it in the negative (left) direction. Reversing the direction of this B field, similarly to the case of FIG. 13A and 13B, will produce the same F(x) force in the same direction. FIG. 14B shows the field in the Z axis, B(z) 710, which does not change its slope at the capsule location 713. B(x) 711 however has the same slope than FIG. 13C shows for the Z axis 714. With the particular selection of the capsule superconductive cylinder's base radius and cylinder high ratios, the forces on the capsule are very closely identical to the forces in FIGS. 13A, 13B and 13C (˜1 Newton).

Having established the field polarity immunity in FIG. 13A through FIG. 14B, we can also prove that superimposition of these fields from different axis will produce directional forces defined by the magnitude and angle of the externally generated fields. FIG. 15A shows a general case for having X and Z components acting on the capsule. The resulting F(x) and F(z) components both levitate (lift) the capsule as well as move it in ‘−X’ direction. The corresponding field strength in X and Z directions are shown in FIG. 15B, for this case.

B(z) 820 is decreasing with the same slope along the Z axis as B(x) 821 is increasing along the X axis. Again, at the capsule, the fluxes are expelled producing the gradients across the superconductive vessel with the commensurate forces driving the capsule upward as well as sideways. This case should suffice to demonstrate the concepts and the generalization for 3D control various combinations of field slopes and gradient directions.

The invention is described herein with reference to certain embodiments, but it is understood that the invention can be embodied in many different forms and should not be construed as limited to the embodiments set forth herein. For example, the embodiments herein disclose that the superconductive material is to be cooled below the critical temperature to attain superconductive characteristics. However, alternate superconducting materials, such as room temperature superconductive material if available, would work equally well, as long as the magnetic phenomena are exhibited by the room temperature superconductive materials. Furthermore, the capsule could be attached to a tether such that the capsule could be removed in the event that the natural muscular (peristaltic) movement of the digestive tract does not expel the capsule, or if the capsule is deployed in a cavity or lumen wherein the capsule must be manually removed. Furthermore, the capsule is not limited to being deployed in humans; the capsule can also be deployed in animals. Therefore, the spirit and scope of the invention should not be limited to any particular combination of elements in the versions described above.

We claim:

1. A magnetically guided capsule, comprising:
a longitudinal housing comprising a first end a second end;
a plurality of enclosures, said first end and said second end adapted to receive one of said plurality of enclosures;
a separately formed vessel adapted to be disposed within said longitudinal housing, said vessel comprising:
at least one superconductive material housed within said vessel, wherein the temperature of said vessel is reduced to a pre-cooled temperature, wherein said pre-cooled temperature is less than the critical temperature of said at least one superconductive material, such that said at least one superconductive material exhibits superconductive Meissner-effect repelling diamagnetic characteristics; and
an insulation material adapted to provide thermal insulation to said at least one superconductive material, wherein said insulation material provides sufficient thermal insulation to said at least one superconductive material such that said at least one superconductive material retains superconducting characteristics;
wherein said vessel is disposed within said longitudinal housing after the temperature of said vessel reaches said pre-cooled temperature;
said capsule adapted to be displaced using supermagnetic repulsion in response to a plurality of externally generated magnetic fields.

2. The magnetically guided capsule of claim 1, wherein said at least one superconductive material is comprised of anisotropic high temperature superconductors.

3. The magnetically guided capsule of claim 2, wherein said at least one superconductive material is comprised of yttrium barium copper oxide.

4. The magnetically guided capsule of claim 1, wherein said vessel is cryogenically cooled to said pre-cooled temperature.

5. The magnetically guided capsule of claim 1, wherein said insulation material further comprises a mirror layer to reflect heat.

6. The magnetically guided capsule of claim 1, wherein said insulation material provides sufficient heat transfer insulation such that said capsule retains superconducting characteristics for at least fifteen minutes.

7. The magnetically guided capsule of claim 1, wherein said insulation material provide sufficient heat transfer insulation such that said capsule retains superconducting characteristics for at least thirty minutes.

8. The magnetically guided capsule of claim 1, wherein said insulation material is comprised of a plurality of insulation layers.

9. The magnetically guided capsule of claim 1, wherein the critical temperature of said at least one superconductive material is 77K and said pre-cooled temperature is 55K.

10. The magnetically guided capsule of claim 1, wherein said vessel is encased in a vacuum prior to being disposed within said housing.

11. The magnetically guided capsule of claim 1, further comprising a camera, said camera adapted to photograph its environment.

12. The magnetically guided capsule of claim 11, further comprising at least one light source to illuminate its environment.

13. The magnetically guided capsule of claim 1, further comprising a wireless transmitter, wherein said wireless transmitter is adapted to transmit captured data to an external receiver.

14. The magnetically guided capsule of claim 1, further comprising a container to store a drug or reactive agent, and a medical delivery device to administer said drug or reactive agent.

15. The magnetically guided capsule of claim 14, wherein said medical delivery device comprises an injection mechanism to deliver said drug or reactive agent.

16. The magnetically guided capsule of claim 14, wherein said medical delivery device comprises a spray mechanism to deliver said drug or reactive agent.

17. The magnetically guided capsule of claim 1, wherein said at least one superconductive material comprises a superconductive ring, disk, plate, dome or a combination thereof.

18. The magnetically guided capsule of claim 1, wherein said externally generated magnetic field displaces said capsule due to the diamagnetic properties of said at least one superconductive material.

19. The magnetically guided capsule of claim 1, wherein a plurality of electromagnetic coils generates said plurality of externally generated magnetic fields.

20. The magnetically guided capsule of claim 19, wherein said plurality of externally generated magnetic fields exert a gradient force and a rotational torque on said at least one superconductive material such that said capsule can be directed by altering said plurality of externally generated magnetic fields.

21. The magnetically guided capsule of claim 20, wherein each of said plurality of electromagnetic coils are independently controlled to produce said gradient force and said rotational torque required to utilize the repelling Meissner-effect forces of said at least one superconductive material to obtain full 6 degree of freedom location and orientation control.

22. The magnetically guided capsule of claim 1, wherein said capsule is ingestible, such that said capsule is adapted to be deployed and navigated through a lumens, cavity or chamber of a human body.
